(19) United States
(12) Patent Application Publication
(10) Pub. No.: US 2012/0054906 A1
(43) Pub. Date: Mar. 1, 2012

(54) Method for Preparation and Purification of Recombinant Proteins

(76) Inventors: Sun Lee, Daejon (KR); Jae-Geun Yoo, Daejon (KR); Suxo Chang, Suwon-shi (KR)

(21) Appl. No.: 10/593,315

(22) PCT Filed: Apr. 2, 2004
(86) PCT No.: PCT/KR2004/000776

§ 371 Date: Sep. 18, 2006

(30) Foreign Application Priority Data
Mar. 16, 2004 (KR) 10-2004-0017691

(51) Int. Cl. A01H 5/00; C12N 15/00; C07K 14/00; C12N 15/82; C12P 21/04; C07H 21/04

(52) U.S. Cl. 800/278; 536/23.6; 435/320.1; 800/298; 435/69.1; 530/350; 530/412

(57) ABSTRACT

The present invention relates to a method for the production, isolation, and purification of a recombinant protein, more particularly, to a method for isolating and purifying a foreign protein stably using Anti-Freeze Protein (AFP), thereby producing the protein. The present invention provides a method for the production, isolation and purification of a foreign target protein using its recombinant protein containing AFP, and a construct, an expression vector, a transformant and a recombinant protein. The recombinant protein produced by the present invention shows the biological property and function identical to a naturally occurring protein. Particularly, the present invention is advantageous for the expression and purification of a useful protein.

### BACKGROUND OF THE INVENTION

1. Field of the Invention

The present invention relates to a method for the production, isolation, and purification of proteins, and materials used in the method. More particularly, it relates to a method for isolating and purifying a foreign protein stably using Anti-Freeze Protein (AFP), and thereby producing the protein, and a construct, an expression vector, a transformant and a recombinant protein related to the method.

2. Description of the Related Art

Recently, plants are given attraction as a system for mass production of proteins, since they can be harvested and processed by traditional agricultural techniques, and thereby, a large amount of biomass can be obtained. Furthermore, unlike bacteria used conventionally as a protein-expression system, plants have a eukaryotic protein synthesis pathway wherein posttranslational modification required for the activation of mammal proteins is made, (Cabanes-Macheteau et al., Glycobiolgy 9:365-372 (1999)). Therefore, the proteins expressed in plants are considered to be almost same as proteins expressed in eukaryotic cell, animal cell, in comparison to proteins expressed in prokaryotic cell, bacteria.

Generally, animal cell lines are used for the production of the recombinant proteins derived from animals. However, animal cell lines require high maintaining cost, and mass production and purification of proteins in them are not easy. In order to resolve such problems, E. coli has been used for mass expression. However, the level of the produced polypeptides is low due to the poor yield of gene expression caused by the low transcription or translation efficiency, and so on. In addition, the produced polypeptides are likely to be degradable since it fails to form a stable 3-dimension structure, or they are aggregated into the inactive inclusion body in the cell. There have been attempts to convert the proteins produced in the E. coli into the biologically active glycosylated proteins through the additional secondary modification process. However, it has limited industrial applicability because of the low modification efficiency and the high cost for the process. On the other hand, plant has eukaryotic protein synthesis pathway wherein the post-translation modification essential for the activity of mammal proteins is made.

Therefore, a transgenic plant transformed with a gene encoding a useful protein has been utilized as a system for producing a target protein.

To produce a foreign protein in plant, it is important to, for example, choose a host plant, and design a promoter and a target gene to be introduced into the plant (modification of target gene for expression in plant and removal of intron). It is necessary to provide isolation and purification method which is useful for practical production of the foreign protein considering such requirements. However, until now, methods for the efficient isolation and purification of proteins expressed in plants have not been achieved successfully.

About 80% of the Earth in aspect of the biological environment belongs to a area below 15° C. Long exposure to below freezing point causes cell-freeing in most organisms, and the leakage of cytoplasm and the formation of ice crystal occur, resulting in cell lysis to cell death. However, the organisms such as fishes existing in intense cold area biosynthesize anti-freeze proteins, AFPs capable of inhibiting the formation and growth of ice crystal in their cell, and they can survive under low temperature. Anti-freeze proteins or anti-freeze glycoproteins have been found in fishes, plants, insects, fungi and bacteria living in cold area (Yamashita et al., Biosci. Biotechnol. Biochem., 66(2):239-247, (2002)).

In fishes, one type of anti-freeze glycoproteins (AFGPs) and several type of unglycosylated anti-freeze proteins (AFPs) have been found, and they have been classified into 4 classes on the basis of their amino acid compositions and structures (Tomczak et al., Biophysical J., 82:874-881, (2002)).

Generally, ice crystal grows in the cycle of attaching and freezing of water. Anti-freeze protein inhibits the size-increase of ice crystal by attaching to the surface of ice crystal.

The present invention utilizes the fact that Anti-freeze protein attaches to ice crystals. In the preparation of a target protein according to the recombinant method, it is the object of the present invention to develop the method for isolating and purifying the target protein by fusing anti-freeze protein to the target protein.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF INVENTION

The present inventors have made intensive study to develop a method for expressing a foreign protein in plant and isolating efficiently the expressed protein from the plant. As a result, the inventors have found that when a target gene was expressed in AFP-fused form, the expressed recombinant protein was efficiently purified in virtue of the property of AFP that it attaches to ice.

Accordingly, it is an object of this invention to provide a novel polynucleotide encoding anti-freeze protein.

It is another object of this invention to provide a nucleotide construct constituting an expression vector.

It is still another object of this invention to provide an expression vector comprising the nucleotide construct.

It is further object of this invention to provide a method for preparing a transient transfected plant expressing a recombinant protein transiently.

It is still further object of this invention to provide a transient transfected plant expressing the recombinant protein transiently.

It is other object of this invention to provide a method for preparing a transgenic plant expressing a recombinant protein stably.

It is still other further object of this invention to provide a transgenic plant expressing the recombinant protein stably.

It is further object of this invention to provide a method for producing a recombinant protein by using a transient transfected plant as a bioreactor.

It is still further object of this invention to provide a method for producing a recombinant protein by using a transgenic plant as a bioreactor.

It is other object of this invention to provide a recombinant protein produced by the above-described method.

It is still other object of this invention to provide a method for isolating recombinant protein using AFP.

Other objects and advantages of the present invention will become apparent from examples to follow, appended claims and drawings.

### DETAILED DESCRIPTION OF THIS INVENTION

In one aspect of this invention, there is provided a polynucleotide encoding anti-freeze protein (AFP), comprising a nucleotide sequence represented by SEQ ID NO:1 which is modified to be expressed well in plants.

In another aspect of this invention, there is provided a nucleotide construct composed, in the following order, of a nucleotide sequence encoding anti-freeze protein comprising the nucleotide sequence represented by SEQ ID NO:1, a protease cleavage site, a multiple cloning site comprising sites recognized by plural restriction enzymes, and stop codon.

In still another aspect of this invention, there is provided a nucleotide construct composed, in the following order, of a multiple cloning site comprising sites recognized by plural restriction enzymes, a protease cleavage site, a nucleotide sequence encoding anti-freeze protein comprising the nucleotide sequence represented by SEQ ID NO:1, and stop codon.

A novel polynucleotide encoding AFP of the present invention comprises a nucleotide sequence (SEQ ID NO:1) modified to be expressed optimally in plants without one replacement of the amino acids of the naturally occurring AFP (FIG. 11).

The present polynucleotide was designed to (i) have GC content of more than about 50%, (ii) have codon usage suitable in plant expression and (iii) avoid intron-like sequences in plant. This sequence suitable for plant increases translation rate of a gene (Kusnadi et al., Biotechnol. Prog. 14:149-155 (1998)). More particularly, it is possible that certain sequence in the introduced gene is recognized as an intron sequence, and digested in plant nucleus, resulting in no production of a desired protein. Therefore, the intron-like sequence in the introduced foreign gene is removed.

The present novel polynucleotide is considered to include not only the nucleotide sequence represented by SEQ ID:NO. 1 but also, a nucleotide sequence which have the substantial identity to the nucleotide sequence represented by SEQ ID:NO. 1 and the significant affinity for ice crystals. The phrase “substantial identity” refers to that an nucleotide sequence has at least 90%, preferably at least 95%, most preferably at least 98% amino nucleotide sequence identity, when the nucleotide sequence of the present invention is compared and aligned for maximum correspondence with an nucleotide sequence, as measured using conventional sequence comparison program.

The present inventors have developed a vector by the insertion of AFP-coding sequence into a conventional vector and the developed vector allows us to isolate and purify a foreign protein expressed in plant conveniently. Until now, a system using AFP has not been reported.

In a preferred embodiment of the present invention, the multiple cloning site comprises at least two recognition sites selected from the group consisting of NcoI, XbaI and BamHI and most preferably, it comprises NcoI, XbaI and BamHI recognition sites.

In the present construct, the protease cleavage site includes any specific sequence recognized by a protease, and most preferably, it is enterokinase or thrombin cleavage site.

The stop codon used in the present invention is TAA, TGA or TAG, and most preferably, TAG.

The present construct is preferably constructed in the following order: 5′-AFP coding sequence-protease cleavage site-multiple cloning site-stop codon-3′. In this case, AFP is linked to N-end of the protein encoded by the foreign sequence inserted into multiple cloning site (FIG. 1a). The following order is a alternative one, 5′-multiple cloning site-AFP coding sequence-protease cleavage site-stop codon-3′. In this case, AFP is linked to C-end of the protein encoded by the foreign sequence (FIG. 1b).

According to the most preferable embodiment, the nucleotide construct comprises the nucleotide sequence represented by SEQ ID:NO 2 or 3.

In a preferred embodiment of the present invention, a structure gene encoding a foreign target protein is inserted into the multiple cloning site. The structural gene may be determined depending on traits of interest. Exemplified structural gene may include but not limited to genes for herbicide resistance (e.g. glyphosate, sulfonylurea), viral resistance, vermin resistance (e.g., Bt gene), resistance to environmental extremes (e.g. draught, high or low temperature, high salt conc.), improvement in qualities (e.g. increasing sugar content, retardation of ripening), exogenous protein production useful as drug (EGF, antigen or antibody to various diseases, insulin) or cosmetic raw material (e.g. albumin, antibiotic peptide).

In another aspect of this invention, there is provided a vector for plant expression, which comprises: (i) the above-described nucleotide construct; (ii) a promoter that functions in plant cells to cause the production of an RNA molecule operably linked to the nucleotide construct of (i); and (iii) a 3′-nontranslated region that functions in plant cells to cause the polyadenylation of the 3′-end of said RNA molecule.

According to a preferred embodiment of the present invention, the above-described nucleotide construct is advantageous for the preparation of vectors for plant expression.

According to a preferred embodiment of the present invention, where the expression vector is constructed for a plant cell, numerous plant-functional promoters known in the art may be used, including the cauliflower mosaic virus (CaMV) 35S promoter, the nopaline synthetase (nos) promoter, the Figwort mosaic virus 35S promoter, the sugarcane bacilliform virus promoter, the commelina yellow mottle virus promoter, the light-inducible promoter from the small subunit of the ribulose-1,5-bis-phosphate carboxylase (ssRUBISCO), the rice cytosolic triosephosphate isomerase (TPI) promoter, the adenine phosphoribosyltransferase (APRT) promoter of Arabidopsis, and octopine synthase promoters.

Regarding the term “operably linked”, typically gene expression is placed under the control of certain regulatory elements including promoters, tissue specific regulatory elements, and enhancers. Such a gene is said to be “operably linked to” the regulatory elements.

According to a preferred embodiment of the present invention, the 3′-non-translated region causing polyadenylation in this invention may include that from the nopaline synthase, gene of Agrobacterium tumefaciens (nos 3′ end) (Bevan et al., Nucleic Acids Research, 11(2):369-385 (1983)), that from the octopine synthase gene of Agrobacterium tumefaciens, the 3′-end of the protease inhibitor I or II genes from potato or tomato, the CaMV 35S terminator.

The vector may alternatively further carry a gene coding for reporter molecule (e.g. luciferase and P-glucuronidase). The vector may contain antibiotic (e.g. neomycin, carbenicillin, kanamycin, spectinomycin and hygromycin) resistance genes (e.g. neomycin phosphotransferase (nptII), hygromycin phosphotransferase (hpt) as selective markers.

According to the present invention, the plant introduced with the vector for plant expression can be prepared by two ways: transient transfected plant and transgenic plant.

The term “transient transfected plant” refers to that the foreign gene introduced into the plant is not transmitted to the next generation of the plant. Generally, the foreign gene is not integrated into the host chromosome in the transient transfected plant.

On the contrary, the term “transgenic plant” refers to the plant wherein the introduced foreign gene is transmitted to the next generation. In the transgenic plant, the foreign gene is integrated into host, and becomes a genetic repertoire of host cell. It is transmitted stably the next generation.

Accordingly, in other aspect of the present invention, it is provided a method for preparing a transient transfected plant, which comprises the steps of: (a) introducing the plant expression vector according to the present invention into a plant cell; and (b) confirming whether the gene has been introduced into said plant cell.

In still other aspect of the present invention, it is provided a transient transfected plant prepared by the above-described method, expressing the plant expression vector transiently.

In further still other aspect of the present invention, it is provided a method for producing a recombinant protein, which comprises the steps of: (a) introducing the plant expression vector according to the present invention into a plant cell; (b) confirming whether the gene has been introduced into said plant cell; and (c) obtaining the recombinant protein from a plant comprising the plant cell introduce with the gene.

In the present method, transient transfection of plant cell can be performed according to a conventional method known to the art (Rainer Fisher et al., Biotechnol. Appl. Biochem., 30:113-116 (1999)). Since transient gene expression in plant allows us to confirm the expression of a target protein rapidly in comparison to transgenic plant, transient gene expression in plant is useful for confirming whether a target protein functions normally or not, in advance of a mass production with stably transformed plant.

For this reason, the present inventors expressed, isolated, and purified foreign proteins using the transient gene-expression method to produce the biologically active foreign proteins in a mass scale.

The transient gene-expression method is useful for determining the function-maintenance and stable expression of the target gene before a transgenic plant is prepared for the mass production of the target protein. Thereby time and cost can be saved. Particularly, in the transformant prepared by a method for the stable transformation, ‘chromosomal positional effect’ depending on the position in which the foreign gene is inserted, is reported. However, since transient transformation can avoid such effect, it is very useful for the expression and isolation of a foreign gene.

There are three representative methods for introducing a foreign gene into a plant cell in the transient transfection method: particle bombardment wherein naked DNA is coated on a particle and it is introduced (Christou, P., Trends Plant Sci. 1:423-431 (1996)), agroinfiltration wherein agrobacterium harvoring expression vector is introduced into plant tissue by vacuum infiltration etc. (Kapila et al., Plant Sci., 122:101-108 (1996)), and viral vectors method wherein a modified plant viral vector is used (Scholthof, H. et al., Annu. Rev. Phytopathol. 34:299-323 (1996)).

The above three methods show different transformation efficiencies. In particle bombardment, generally, DNA is introduced into only several cells, and DNA should reach to the cell nucleus for transcription. This method is advantageous for verifying the stability of the recombinant protein in advance of the stable transformation, but unsuitable for the mass expression of the recombinant protein.

Agroinfiltration is capable of introducing the target gene into more cells than particle bombardment, and T-DNA containing the target gene is introduced actively into the nucleus with the assistance of some bacterial proteins. Introduced T-DNA is not integrated into the chromosome of host cell and it is not expressed continuously in this method, too. It exists independently in the nucleus, and the expression of target protein is transient. Therefore, this method is suitable for producing protein as much as sufficient to study the stability and function of the protein rapidly.

In viral vector method, a target protein is introduced into the genome of viral plant pathogen, and a strong promoter regulates it. Where a plant is transfected with the recombinant viral vector containing a foreign gene, the introduced gene is amplified in high efficiency during the replication of plant virus and the gene is expressed. However, because viral vector is not applicable for the target protein over 30 kDa, it is not suitable for a large gene.

Therefore, agroinfiltration is preferred in the present invention. Agroinfiltration is usually carried out with leaf.

More preferably, agroinfiltration is carried out using Agrobacterium tumefaciens-binary vector system.

In the above embodiment, suitable leaf include any one derived from a germinated seed, preferably the leaf formed before a flower stalk growths, most preferably the leaf not too young or mature. The younger is it, the higher the level expression, but the tissue is subject to chlorosis or die. Since the tissue becomes expanded in a mature leaf, the introduction of Agrobacterium is convenient. However, the expression level is lower. Seed germination is performed using appropriate mediums under suitable dark/light conditions.

The introduction of gene into plant cells is carried out with Agrobacterium tumefaciens harboring Ti plasmid (Depicker, A. et al., In Genetic Engineering of Plants, Plenum Press, New York (1983)). More preferably, binary vector system such as pBinl9, pRD400, pRD320 and pHS737 is used for transformation (An et al., “Binary vectors” In Plant Gene Res. Manual, Martinus Nijhoff Publisher, New York (1986); Jain, et al., Biochem. Soc. Trans. 28:958-961 (2000)).

The introduction of gene into the plant cell of leaf with Agrobacterium tumefaciens involves procedures known in the art. Most preferably, the introduction of gene involves infiltrating Agrobacterium tumefaciens culture into leaf tissue.

Infiltration may be carried out with vacuum method or with injection method. An exemplified example by using vacuum method is as follows. A Leaf of plant is immersed in Agrobacterium tumefaciens culture and a vacuum is applied for a short time. After releasing vacuum as rapid as possible, the leaf is washed with sterilized water and the leaf is placed on the wetted paper in a manner that the front side of it faces the paper. The leaf is kept at 22° C. for about 16 hr under light, and then, expression of target protein is confirmed about 2-3 hr after.

An exemplified example by using injection method is as follows. Agrobacterium tumefaciens culture in syringe is treated to the back of leaf. The leaf is cultivated for about 3-5 days, and then, expression of target protein is confirmed.

Preferably, acetosyringone is added to the Agrobacterium tumefaciens culture to facilitate the infiltration of Agrobacterium into plant.

The introduction of target gene into plant prepared according to the present invention may be confirmed using procedures known in the art. For example, using DNA sample from the gene-introduced tissue, PCR is carried out to reveal exogenous gene incorporated into the genome of the plant. Alternatively, Northern or Southern Blotting may be performed for confirming the introduction of the gene as described in Maniatis et al., Molecular Cloning, A Laboratory Manual, Cold Spring Harbor Laboratory, Cold Spring Harbor, N.Y. (1989). Where the vector harbored in Agrobacterium tumefaciens contains a gene encoding β-glucuronidase, a portion of the gene introduced leaf is immersed in substrate solution such as X-gluc (5-Bromo-4-Chloro-3-Indole-β-D-Glucuronic Acid) so that calorimetric reaction may be observed to confirm the occurrence of the introduction of target gene (Jefferson, Plant Mol. Biol. Rep., 5:387 (1987)).

According to the present method, transient transfected plants enable us to produce a foreign gene rapidly at low cost.

A foreign protein can be obtained from the gene-introduced plant, or the transient transfected plant (e.g., leaf). The extract from the plant can be treated with the conventional purification procedure. In this invention, purification methods conventionally used in the art may be employed. For example, various methods including solubility fractionation by use of ammonium sulfate or PEG, size differential filtration and column chromatography (based on size, net surface charge, hydrophobicity or affinity) are available and usually the combination of the methods is used for purification.

According to the most preferred embodiment, the protein expressed according to the present invention has AFP at its N-end or C-end. Therefore, the desired foreign protein can be isolated and purified rapidly and conveniently by using ice-crystal.

Referring to the specific Examples herewith, the isolation and purification method can be described as follows:

### (1) Isolation and Purification of Protein in a Mass Scale Using Ice-Filled Column

Extracting buffer (e.g., sucrose, Hepes, MgCl₂ and DTT) containing a protease inhibitor is added to the plant tissue introduced with AFP-GFP (or GFP-AFP gene) and grinded well. The extract is centrifuged, followed by collecting supernatant. The supernatant is loaded on the column containing ice crystals, and the column is shaken to allow AFP-GFP (or GFP-AFP) protein to be attached to the ice crystals. The ice crystals are collected by centrifugation. The collected ice crystals are dissolved in the same amount of phosphate buffer, and the released proteins are concentrated.

### (2) Isolation and Purification of Protein in a Mass Scale Using Ice-Nucleation Material

Extracting buffer containing a protease inhibitor is added to the plant tissue introduced with AFP-GFP (or GFP-AFP gene) and grinded well. The extract is centrifuged, followed by collecting supernatant. The supernatant is super-cooled under stirring in cold-bath. An ice-nucleation material (e.g., AgI or alive or dead Pseudomonas syringae) is added to the supernatant and it is stirred continuously to allow AFP-GFP protein (or GFP-AFP protein) to be attached to the ice crystals until ⅔ of the supernatant is formed into ice-crystals. The ice formed crystals are collected by centrifugation. The collected ice crystals are dissolved in the same amount of phosphate buffer, and the released proteins are concentrated.

### (3) Isolation and Purification of Protein in a Mass Scale Using Hypertonic Solution

Extracting buffer containing a protease inhibitor of protein-hydrolysis enzyme is added to the plant tissue introduced with AFP-GFP gene (or GFP-AFP gene) and grinded well. According to a preferred embodiment of the present invention, Sucrose is preferred for the preparation of the hypertonic solution. However, various monosaccharides, disaccharides, polysaccharides or sugar-alcohol may be used. The concentration of the hypertonic solution can be chosen depending on the intention, and 5%-50% is preferred. The extract is centrifuged, followed by collecting supernatant. The supernatant is stirred continuously to allow AFP-GFP protein (or GFP-AFP protein) be attached to the ice crystals in cold-bath until ⅔ of the supernatant was formed into ice-crystals. The ice formed crystals are collected by centrifugation. The collected ice crystals are dissolved in the same amount of phosphate buffer, and the released proteins are concentrated.

### (4) Isolation and Purification of Protein in a Mass Scale Using Freeze-Control Device

Extracting buffer containing a protease inhibitor is added to the plant tissue introduced with AFP-GFP gene (or GFP-AFP gene) and grinded well. The extract is centrifuged, followed by collecting supernatant. The supernatant is placed in an ice maker and frozen until ⅔ of the supernatant is formed into ice-crystals. Unfrozen solution is vented and extracting buffer cooled to 0° C. is added. The ice crystals are washed to remove materials except the target protein. The ice crystals are dissolved in the same amount of phosphate buffer, and the released proteins are concentrated. According to a preferred embodiment of the present invention, the ice maker is equipped with a low temperature controller and a stirrer for controlling freezing-rate.

Alternative approach to prepare the plant introduced with a gene encoding a foreign protein is to prepare the transgenic plant expressing a foreign protein stably.

Accordingly, in other aspect of this invention, there is provided a method for preparing transgenic plant expressing a foreign protein stably, which comprises the steps of: (a) transforming a plant cell with the vector of the present invention; (b) selecting a transformed plant cell; and (c) regenerating the transformed plant cell to obtain a transgenic plant.

In still other aspect of this invention, there is provided a transgenic plant prepared by the above-described method, expressing the foreign protein stably.

In further aspect of this invention, there is provided a method for producing a recombinant foreign protein, which comprises the steps of: (a) transforming a plant cell with the vector of the present invention; (b) selecting a transformed plant cell; (c) regenerating the transformed plant cell to obtain a transgenic plant; and (d) recovering the recombinant foreign protein from the transgenic plant.

The transformation of plant cells may be carried out according to the conventional methods known one of skill in the art, including electroporation (Neumann, E. et al., EMBO J., 5 1:841 (1982)), particle bombardment (Yang et al., Proc. Natl. Acad. Sci., 87: 9568-9572 (1990)) and Agrobacterium-mediated transformation (U.S. Pat. Nos. 5,004,863, 5,349,124 and 5,416,011). Among them, Agrobacterium-mediated transformation is the most preferable. Agrobacterium-mediated transformation is generally performed with leaf disks and other tissues such as cotyledons and hypocotyls. This method is the most efficient in dicotyledonous plants.

The selection of transformed cells may be carried out with exposing the transformed cultures to a selective agent such as a metabolic inhibitor, an antibiotic and herbicide. Cells which have been transformed and have stably integrated a marker gene conferring resistance to the selective agent will grow and divide in culture. The exemplary marker includes, but not limited to, a glyphosphate resistance gene and a neomycin phosphotransferase (nptII) system.

The development or regeneration of plants from either plant protoplasts or various explants is well known in the art (Bhojwani et al., Plant Tissue Culture: Theory and Practice, Elsevier Science, New York, (1983); and Lindsey, Ed., Plant Tissue Culture Manual, Kluwer Academic Publishers, Dordrecht, The Netherlands, (1991)). The resulting transgenic rooted shoots are planted in an appropriate plant growth medium. The development or regeneration of plants containing the foreign gene of interest introduced by Agrobacterium may be achieved by methods well known in the art (U.S. Pat. Nos. 5,004,863, 5,349,124 and 5,416,011).

Meanwhile, the present inventors have made attempts to develop novel transformed plants such as Nicotiana tabacum, Cucumis melo, Curcumis sativa, Citrullus vulgaris and Brassica campestris and as a result, have established the most efficient methods for the transformation of certain plant. Such methods have been filed for patent application (PCT/KR02/01461, PCT/KR02/01462 and PCT/KR02/01463).

According to a preferred embodiment, the plant to be transformed is Nicotiana tabacum, Cucumis melo, Curcumis sativa, Citrullus vulgaris and Brassica campestris.

In the present invention, the preferred transformation is carried out using Agrobacterium system, more preferably, Agrobacterium tumefaciens-binary vector system.

In this invention, the preferred explant for transformation includes any tissue derived from seed germinated. It is preferred to use cotyledon and hypocotyl and the most preferred is cotyledon. Seed germination may be performed under suitable dark/light conditions using an appropriate medium. Transformation of plant cells derived is carried out with Agrobacterium tumefaciens harboring Ti plasmid (Depicker, A. et al., Plant cell transformation by Agrobacterium plasmids. In Genetic Engineering of Plants, Plenum Press, New York (1983)).

More preferably, binary vector system such as pBinl9, pRD400 and pRD320 is used for transformation (An, G. et al., Binary vectors “In Plant Gene Res. Manual, Martinus Nijhoff Publisher, New York (1986)). The binary vector useful in this invention carries: (i) a promoter capable of operating in plant cell; (ii) a structural gene operably linked to the promoter; and (iii) a polyadenylation signal sequence. The vector may alternatively further carry a gene coding for reporter molecule (for example, luciferase and ss-glucuronidase). Examples of the promoter used in the binary vector include, but not limited to, cauliflower mosaic Virus 35S promoter, 1′promoter, 2′promoter and promoter nopaline synthetase (nos) promoter.

Inoculation of the explant with Agrobacterium tumefaciens involves procedures known in the art. Most preferably, the inoculation involves immersing the cotyledon in the culture of Agrobacterium tumefaciens to coculture. Agrobacterium tumefaciens is infected into plant cells.

The explant transformed with Agrobacterium tumefaciens is regenerated in a regeneration medium, which allows successfully the regeneration of shoots. The transformed plant is finally produced on a rooting medium by rooting of regenerated shoots.

The transformed plant produced according to the present invention may be confirmed using procedures known in the art. For example, using DNA sample from tissues of the transformed plant, PCR is carried out to elucidate exogenous gene incorporated into a genome of the transformed plant. Alternatively, Northern or Southern Blotting may be performed for confirming the transformation as described in Maniatis et al., Molecular Cloning, A Laboratory Manual, Cold Spring Harbor Laboratory, Cold Spring Harbor, N.Y. (1989).

The foreign protein expressed in the transformant may be provided from the tissues derived from various transformed organs (stem, leaf, root, fruit and seed) and be obtained by purifying the extracts of the tissues by using the above-described method for purifying the foreign protein from the gene-introduced plant, or transient transfected plant tissue (for example: leaf).

In other aspect of this invention, there is provided a method for isolating a recombinant protein using the property of AFP that it attaches to ice, which comprises the step of; (a) contacting to ice crystal a recombinant fusion protein comprising target protein and AFP; and (b) recovering the ice crystal to which the recombinant protein is attached.

According to a preferred embodiment of the present invention, said AFP is derived from plants, fungi or fishes (see, U.S. Pat. No. 6,096,867 and WO 99/00493).

According to a preferred embodiment of the present invention, said AFP used for isolation of the recombinant protein corresponds to not only the full length of amino acid sequence of AFP, but also the domain sequence functioning as ice-attaching domain. The domain sequence is enough to provide the same result that is achieved with the full length.

According to a preferred embodiment of the present invention, contacting the recombinant protein to ice-crystal is carried out by loading the solution containing the recombinant protein on ice-filled column or by adding an ice-nucleation material to the solution containing the recombinant protein to form ice-crystal.

According to a preferred embodiment of the present invention, recovering ice crystals is carried out in accordance with the conventional method used for the isolation of a solid material from a solution (for example, filtration or centrifugation), and the preferred method is centrifugation.

According to a preferred embodiment of the present invention, the recombinant protein is produced by the method for preparing a transient transfected plant or a transgenic plant expressing the recombinant protein, which comprises the steps of; (a) preparing an expression vector comprising a construct in which a nucleotide sequence encoding AFP is linked to 5′-end or 3′-end of a nucleotide sequence encoding a target protein and protease cleavage site exists between the target protein-coding sequence and AFP-coding sequence; (b) introducing the expression vector into a host; and (c) selecting a transformed host.

In the preferred embodiment of the present invention, the protease cleavage site is enterokinase cleavage site or thrombin cleavage site.

In the preferred embodiment of the present invention, the expression vector is the expression vector for plant, animal or microorganism. The expression vector may include a conventional vector used for the expression of a foreign protein in plant, animal or microorganism in the art. The vector system of this invention may be constructed according to the known methods in the art as described in Sambrook et al., Molecular Cloning, A Laboratory Manual, Cold Spring Harbor Laboratory Press (2001), which is incorporated herein by reference.

The vector of the present invention may be constructed for use in prokaryotic or eukaryotic host cells. For example, where the vector is constructed for expression in prokaryotic cells, it generally carries a strong promoter to initiate transcription (e.g., pL^{λ} promoter, trp promoter, lac promoter, tac promoter and T7 promoter), a ribosome binding site for translation initiation and a transcription/translation termination sequence. In particular, where E. coli (e.g., HB101, BL21 or DH5α) is used as a host cell, a promoter and operator in operon for tryptophan biosynthesis in E. coli (Yanofsky, C., J. Bacteriol., 158:1018-1024 (1984)) and a leftward promoter of phage λ (pLλ promoter, Herskowitz, I. and Hagen, D., Ann. Rev. Genet., 14:399-445 (1980)) may be employed as a control sequence. Where Bacillus is used as a host cell, a promoter for a gene encoding toxin protein of Bacillus thurigensis (Appl. Environ. Microbiol. 64:3932-3938 (1998); and Mol. Gen. Genet. 250:734-741 (1996)) or other promoters operable in Bacillus may be employed as a control sequence. The vector used in the present invention may be prepared from plasmid (e.g.: pSC101, pGV1162, pACYC177, ColE1, pKT230, pBR322, pUC8/9, pBD9, pHC79, pIJ61, pLAFR1, pHV14, pGEX series, pET series and pUC19), phage (e.g.: λ gt4•λB, λ-Charon, λΔz1 and M13 etc.) or virus (e.g.: SV40) used conventionally in the art.

Where the vector of the present invention is an expression vector constructed for eukaryotic host cell, a promoter derived the genome of mammalian cells (e.g., metallothionein promoter) or mammalian virus (e.g., adenovirus late promoter; vaccinia virus 7.5K promoter, SV40 promoter, cytomegalovirus promoter and tk promoter of HSV) may be used. The vector generally contains a polyadenylation site as a transcription termination sequence.

The expression vector for eukaryotic host cell of the present invention may be chosen from various vectors known to the art: for example, such as YIp5, Ycp19 and bovine papilloma virus.

It is preferable that the expression vector of this invention carries one or more markers which make it possible to select the transformed host, for example, genes conferring the resistance to antibiotics such as ampicillin, gentamycine, carbenicillin, chloramphenicol, streptomycin, kanamycin, geneticine, neomycin, geneticin and tetracycline. Ampicillin or gentamycine is preferred in the aspect of cost.

The hosts useful in cloning and expressing the vector of the present invention are well known to those skilled in the art. For example, E. coli JM109, E. coli BL21, E. coli RR1, E. coli LE392, E. coli B, E. coli X 1776, E. coli W3110, Bacillus subtilis, Bacillus thurigensis, Salmonella typhimurium, Serratia marcescens and various Pseudomonas may be employed.

As eukaryotic cell, yeast (Saccharomyce cerevisiae), insect cell and human cell (e.g., CHO cell lines (Chinese hamster ovary)), W138, BHK, COS-7, 293, HepG2, 3T3, RIN and MDCK cell lines cell) may be used.

The transformation of a host cell can be carried out by a large number of methods known to one skilled in the art. For example, in case of using prokaryotic cells as host, CaCl₂ method (Cohen, S. N. et al., Proc. lVatl. Acac. Sci. USA, 9:21102114 (1973)), Hanahan method (Cohen, S. N. et al., Proc. Matl. Acac. Sci. USA, 9:2110-2114 (1973); and Hanahan, D., J. Mol. Biol., 166:5 S7-580 (1983)) and electroporation (Dower, W. J. et al., Nucleic. Acids Res. 16:6127-6145 (1988))’ can be used for transformation. Also, in case of using eukaryotic cells as host, microinjection (Capecchi, M. R., Cell, 22:479 (1980)), calcium phosphate precipitation (Graham, F. L. et al., virology, 52:456 (1973)), electroporation (Neumann, E. et al., EMBO J., 1:841 (1982)), liposome-mediated transfection (Wong, T. K. et al., Gene, 10:87 (1980)), DEAE-dextran treatment (Gopal, Mol. Cell Biol., 5:1188-1190 (1985)), and particle bombardment (Yang et al., Proc. Natl. Acad. Sci., 87:9568-9572 (1990)) can be use for transformation.

The selection of transformed hosts is carried out by using phenotype conferred by selection marker. For example, if the maker is a resistance gene to certain antibiotics, the cultivation of putative transformants in the medium containing the antibiotics makes it possible to select transformants.

### BRIEF DESCRIPTION OF THE DRAWINGS

FIG. 1a represents the construct comprising Anti-Freeze Protein (AFP) gene or other identically functioning gene located at 5′-end of cloning site (AFPN),

FIG. 1b represents the construct comprising Anti-Freeze Protein gene or other identically functioning gene located at 3′-end of cloning site (AFPC),

FIG. 2a represents the scheme of the restriction map of pB1121AFPN and pB1121AFPC vectors for plant expression,

FIG. 2b represents the scheme of the restriction map of pRD400AFPN and pRD400AFPC vectors for plant expression,

FIG. 3 shows the result of the electrophoresis of pRD400AFPN and pRD400AFPC vectors cloned with gene cassette comprising Anti-Freeze Protein and digested with HindIII and EcoRI (M, 1 kb DNA ladder; lane 1, pRD400AFPN vector digested with HindIII and EcoRI; lane 2, pRD400AFPC vector digested with HindIII and EcoRI; lane 3, AFPN PCR product; [symbol] 4, AFPC PCR product),

FIG. 4 shows the result of electrophoresis of pRD400AFPN vector cloned with GFP gene and digested with HindIII and EcoRI (M, 1 kb DNA ladder; lane 1, GFP gene-containing pRD400AFPN-GFP plasmid digested with HindIII and EcoRI; lane 2, AFP-GFP PCR product),

FIG. 5 shows the expression of GFP in the plant introduced with the vector: A, wild type of Nicotiana benthamiana, and B, plant cells emitting fluorescence generated by GFP expressed in the gene-introduced plant,

FIG. 6 shows the results of the electrophoresis of AFP-GFP protein expressed in the vector-introduced plant, or the transient transfected plant on SDS-polyacrylamide gel and the Western blotting thereof (A, electrophoresis photograph; b, Western blot photograph; M, molecular marker; lane 1, proteins from wild type of Nicotiana benthamiana; and lane 2, GFP expressed in the gene-introduced plant)

FIG. 7 shows the results of isolation and purification of the recombinant AFP-GFP protein using ice-filled column (A, photograph of electrophoresis performed on SDS-polyacrylamide gel: M, molecular marker; lane 1, proteins from wild type of Nicotiana benthamiana; and lane 2, AFP-GFP protein expressed in transient transfected plant and isolated and purified by using ice-filled column, B, photograph of Western blot of A: M, molecular marker; lane 1, proteins from wild type of Nicotiana benthamiana; and lane 2, AFP-GFP protein expressed in transient transfected plant and isolated and purified by using ice-filled column),

FIG. 8 shows the results of isolation and purification of the recombinant AFP-GFP protein using a ice-nucleation material (A, photograph of electrophoresis performed on SDS-polyacrylamide gel: M, molecular marker; lane 1, proteins from wild type of Nicotiana benthamiana; lane 2, AFP-GFP protein expressed in transient transfected plant and isolated and purified by the ice-nucleation method with AgI; and lane 3, AFP-GFP protein expressed in transient transfected plant and isolated and purified by the ice-nucleation method with Pseudomonas syringae, B, photograph of Western blot of A: M, molecular marker; lane 1, proteins from wild type of Nicotiana benthamiana; lane 2, AFP-GFP protein expressed in transient transfected plant and isolated and purified by the ice-nucleation method with AgI; and lane 3, AFP-GFP protein expressed in transient transfected plant and isolated and purified by the ice-nucleation method with Pseudomonas syringae),

FIG. 9 shows the results of isolation and purification of the recombinant AFP-GFP protein using hypertonic solution (A, photograph of electrophoresis performed on SDS-polyacrylamide gel: M, molecular marker; lane 1, proteins from wild type of Nicotiana benthamiana; lane 2, AFP-GFP protein isolated and purified by using the extracting solution containing 2-250 mM sucrose; and lane 3, AFP-GFP protein isolated and purified by using the extracting solution containing 3-15% sucrose (hypertonic solution), B, photograph of Western blot of A: M, molecular marker; lane 1, proteins from wild type of Nicotiana benthamiana; lane 2, AFP-GFP protein isolated and purified by using the extracting solution containing 2-250 mM sucrose; and lane 3, AFP-GFP protein isolated and purified by using the extracting solution containing 3-15% sucrose (hypertonic solution),

FIG. 10 shows the results of isolation and purification of the recombinant AFP-GFP protein using a freeze-controlling inducible material (A, photograph of electrophoresis performed on SDS-polyacrylamide gel: M, molecular marker; lane 1, proteins from wild type of Nicotiana benthamiana; and lane 2, AFP-GFP protein expressed in transient transfected plant and isolated and purified by using a freeze-control device; B, photograph of Western blot of A: M, molecular marker; lane 1, proteins from wild type of Nicotiana benthamiana; and lane 2, AFP-GFP protein expressed in transient transfected plant and isolated and purified by using a freeze control device, and

FIG. 11 represents 42 amino acid of naturally occurring AFP.

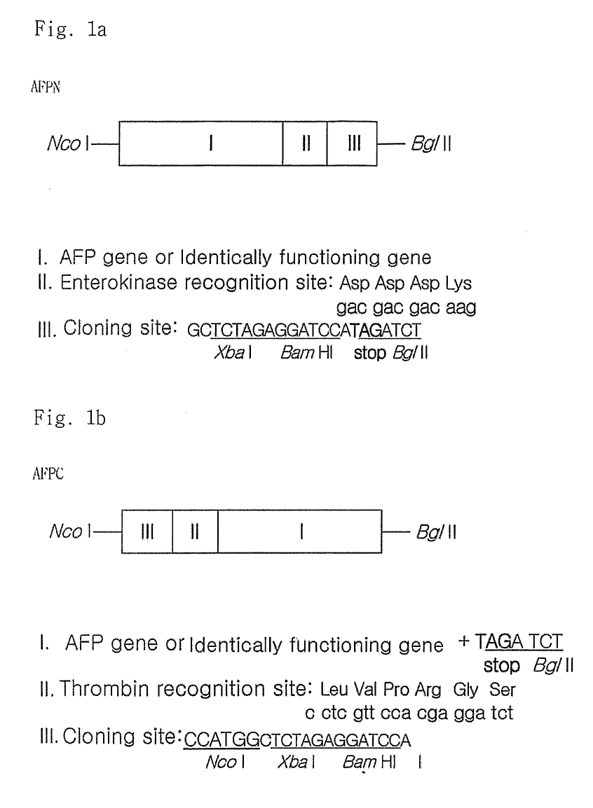

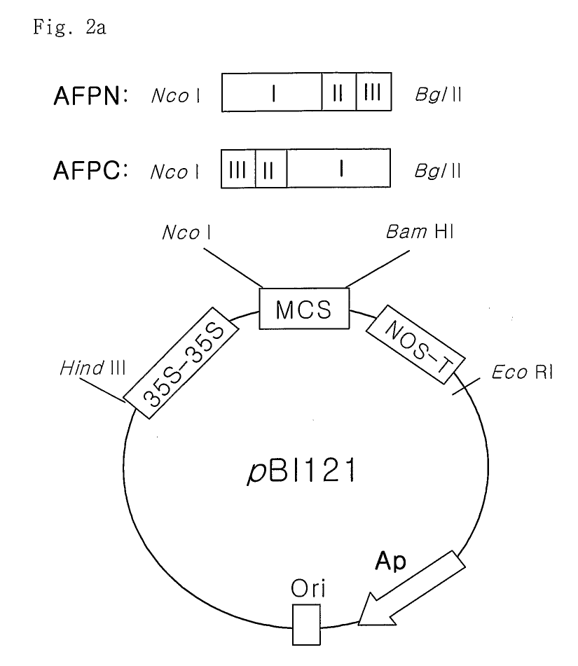

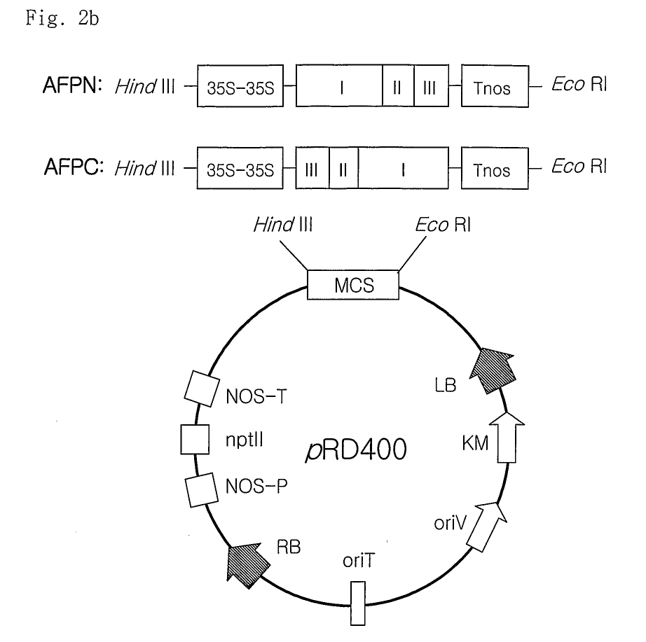

The following specific examples are intended to be illustrative of the invention and should not be construed as limiting the scope of the invention as defined by appended claims.

### EXAMPLES

### Example

### Preparation of Novel Gene Encoding AFP

An optimal nucleotide sequence suitable in plant expression and microorganisms was designed in such a manner that the nucleotide sequence codes for naturally occurring AFP consisting of 42 amino acids, and different from known nucleotide sequences coding AFP. The present novel gene was designed to have GC content of more than about 50%, have codon usage suitable in expression in plant and avoid intron-like sequences in plant. The nucleotide sequences thus newly designed were chemically synthesized in plant Biotechnology Institute (PBI), National Research Centre (SK, Canada). The DNA encoding AFP are indicated in SEQ ID Nos:1.

### Example 1

### Preparing DNA Domain Comprising Nucleotide Sequence of AFP

A nucleotide sequence was designed to comprise AFP for protein purification, Enterokinase cleavage site for protein separation, multiple cloning site for cloning foreign genes, and stop codon. The Multiple cloning site has NcoI, XbaI and BamHI restriction sites, and TGA stop codon for the termination of protein synthesis (see, SEQ ID. NO 2—NcoI, AFP typeI+Enterokinase+cloning site (XbaI and BamHI)+TAG+BglII; 5′-CC ATGGCTCT AGAGGATCCC CTCGTTCCAC GAGGATCTAT GGATGCTCCA GCTAAAGCAG CAGCAAAAAC AGCTGCAGAT GCAAAAGCTG CTGCTGCTAA AACTGCAGCA GATGCATTAG CTGCTGCTAA TAAAACTGCA GCAGCTGCTA AAGCAGCTGC AAAA TAGA TCT-3′, AFPN). The nucleotide sequences thus designed were chemically synthesized in PBI, National Research Centre (SK, Canada).

A nucleotide sequence was designed to comprise multiple cloning site for cloning foreign genes, Thrombin cleavage site for protein separation, AFP for protein purification, and stop codon. The Multiple cloning site has NcoI, XbaI and BamHI restriction sites, and the end region of AFP has TGA stop codon for the termination of protein synthesis (see, SEQ ID. NO 3—NcoI+cloning site (XbaI+Bam HI)+Thrombin+AFP typeI+TAG+BglII: 5′-CC ATGGATGCTC CAGCTAAAGC AGCAGCAAAA ACAGCTGCAG ATGCAAAAGC TGCTGCTGCT AAAACTGCAG CAGATGCATT AGCTGCTGCT AATAAAACTG CAGCAGCTGC TAAAGCAGCT GCAAAAGACG ACGACGACAA GGCTCTAGAG GATCCATAGA TCT-3′, AFPC). The nucleotide sequences thus designed were chemically synthesized in PBI, National Research Centre (SK, Canada).

### Example II

### Preparation of Plant Expression Vectors Carrying AFP Gene

In the nucleotide constructs comprising AFP-coding sequence, AFPN consisted of 165 bp, and AFPC consisted of 171 bp. The constructs had NcoI and BglII restriction sites at their 5′- and 3′-ends, respectively. Such design was intended to express the genes in plant by locating 35S promoter for plant expression at 5′-end of AFPN or AFPC and nos terminator at 3′-end of AFPN or AFPC. That is, it was intended to clone the constructs into the cloning sites NcoI and BamHI of pBI121 which allow us to introduce genes or gene cassettes between 35S promoter and nos terminator. NcoI is complementary with NcoI and BglII is with BamHI. Since restriction enzyme recognition sites are removed after each restriction enzyme site was ligated, it is possible to use the cloning sites included in the constructs efficiently.

The gene expression cassette comprising 35S promoter, synthesized 165-bp AFPN gene and nos terminator was digested with HindIII and EcoRI, isolated and purified. To introduce the gene expression cassette into the binary vector for plant expression, pRD400, pRD400 was digested with XbaI and BamHI and purified. After purifying and recovering the corresponding DNAs, the gene expression cassette digested with HindIII and EcoRI, and pRD400 vector digested in the same manner were mixed and ligation buffer (KOSCO, Korea) and T4 ligase (KOSCO, Korea) were added to the mixture to incubate for 1 hr at 16° C. Thereafter, the reaction product was transformed into CaCl₂-treated competent cell (E. coli strain DH5α; Promega, USA) and then the transformed cells with ampicillin resistance were selected by culturing in LB medium containing ampicillin (100 mg/ml).

From the selected clones, plasmid DNAs were isolated according to the alkaline method and the DNAs were amplified according to the polymerase chain reaction using a primer set: forward primer, 5′-CCA TGG ATG CTC CAG CTA-3′ and reverse primer 5′-AGA TCT ATG GAT CCT CTA-3′. The PCR was performed using Taq polymerase (Solgent, Korea) under the following condition: pre-denaturation at 96° C. for 2 min followed by 35 cycles of denaturation at 94° C. for 1 min, annealing at 55° C. for 1 min and extension at 72° C. for 2 min and; followed by final extension at 72° C. for 10 min.

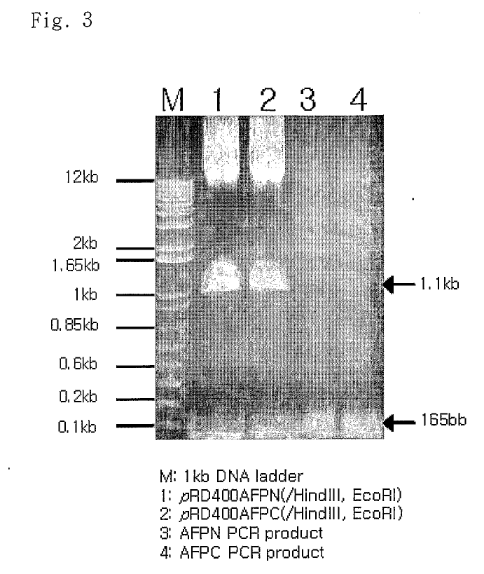

Amplified products were analyzed by electorphoresis on 1.0% agarose gel to verify the existence of insert sequence in plasmids (see FIG. 3). The PRD400 vector cloned with the cassette AFPN comprising AFP was named “pRD400AFPN”.

Additionally, synthesized 171-bp AFPC gene was processed with the same manner as describe above, and the desired recombinant plasmid was obtained. The PCR was performed to verify insert gene AFPC using forward primer 5′-CCA TGG CTC TAG AGG ATC-3′ and reverse primer 5′-ATC TAT TTT GCA GCT GCT-3′.

Amplified products were analyzed by electorphoresis on 1.0% agarose gel to verify the existence of insert sequence in plasmids (see FIG. 3). The PRD400 vector cloned with the cassette AFPC comprising AFP was named “pRD400AFPC”.

### Example 3

### Preparation of Plant Expression Vectors Carrying GFP Gene

To verify the expression, isolation and purification of AFP fusion foreign protein, Green fluorescent protein (GFP) was employed. Recombinant 818 bp GFP gene encoding GFP was cloned into the AFPN-comprising pRD400AFPN vector prepared in Example 2.

pRD400AFPN vector for plant expression and GFP gene (Genbank No. U87974) were digested respectively with XbaI and BamHI, isolated and purified. The digested GFP gene and pRD400AFPN vector were mixed and ligation buffer (KOSCO, Korea) and T4 ligase (KOSCO, Korea) were added to the mixture to incubate for 1 hr at 16° C. Thereafter, the reaction product was transformed into CaCl₂-treated competent cell (E. coli strain DH5α; Promega, USA) and then the transformed cells with ampicillin resistance were selected by culturing in LB medium containing ampicillin (100 mg/ml).

From the selected clones, plasmid DNAs were isolated according to the alkaline method and the DNAs were amplified according to the polymerase chain reaction using a primer set: forward primer, 5′-AGT AAA GGA GAA GAA CTT-3′ and reverse primer 5′-TTT GTA TAG TTC ATC CAT-3′. The PCR was performed using Taq polymerase (Solgent, Korea) under the following condition: pre-denaturation at 96° C. for 2 min followed by 35 cycles of denaturation at 94° C. for 1 min, annealing at 55° C. for 1 min and extension at 72° C. for 2 min and; followed by final extension at 72° C. for 10 min.

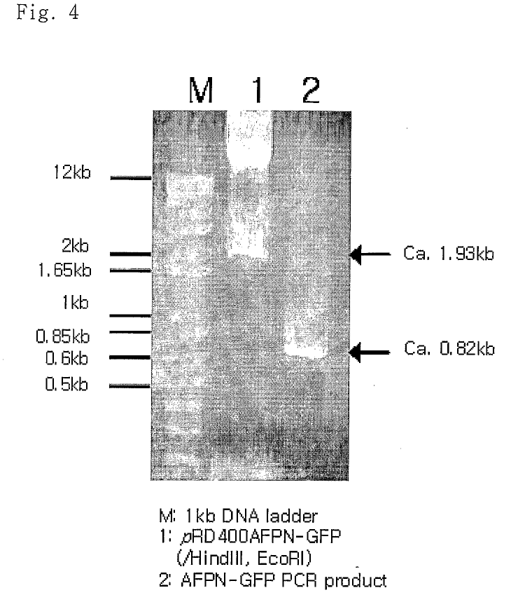

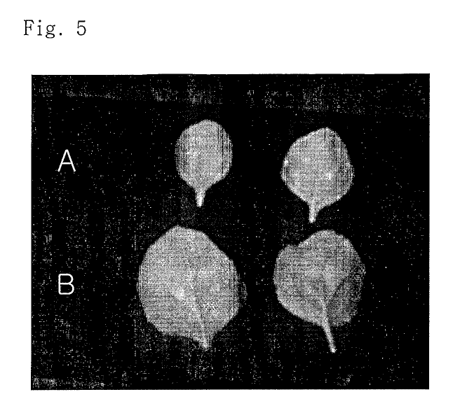

Amplified products were analyzed by electrophoresis on 1.0% agarose gel to verify the cloning of the desired GFP gene in plasmids (see FIG. 4).

### Example 4

### Introduction of Vector for Plant Expression into Plant

### Example 4-1

### Preparation of Agrobacterium tumefaciens GV3101 Culture

The recombinant pRD400AFPN-GFP vector prepared in Example 3 was introduced into Agrobacterium tumefaciens GV3101 (Mp90) (Plant-cell-rep., 15(11) 799-803 (1996)) by means of conjugation. Each of the transformed E. coli cells harboring the recombinant pRD400AFPN-GFP vector and Agrobacterium tumefaciens GV3101 (Mp90) was incubated in LB liquid medium to the exponential stage. The two types of cells were mixed at a ratio of 1:1 in eppendorf tube and centrifuged for 30 sec to concentrate, followed by the stationary culture for 1-2 days at 28° C. The eppendorf tubes were mildly shaken to suspend cells and the suspension was spread on LB solid medium containing 50 mg/l of kanamycin and 30 mg/l gentamicin and incubated for 2 days at 28° C. to select colonies harboring the expression vector (Alt-Morbe et al., J. Bacteriol. 178:4248-4257 (1996)).

Agrobacterium tumefaciens containing pRD400AFPN-GFP vector was inoculated into Lb liquid medium and incubated for 2 days at 28° C. and the culture was used as seed culture.

The sufficiently cultured seeds in 1/50 of the final culture volume were added to LB liquid medium with antibiotics (50 mg/l kanamycin). It was incubated for about 18 hr until showing its absorbance 1.5 at 600 nm. This culture medium was centrifuged at 3500 rpm for 15 min at room temperature and the supernatant was discard. The pellets were suspended in 10 mM MgCl₂ solution containing BA 0.2 ppm and acetosyringone 150 μM. The suspension was left at room temperature over 3 hr and used for plant transfection.

### Example 4-2

### Preparation of Plant Cell and Agroinfiltration

The sterilized seeds of Nicotiana benthamiana were seeded and cultivated over 3 weeks. They were acclimated in the greenhouse. A inoculation medium were prepared according to the method described in Example 4-1 with Agrobacterium tumefaciens GV3101(Mp90) containing pRD400AFPN-GFP vector and filled in 1 ml of a syringe of which the needle was removed (GreenCross Inc.). The inoculation medium was spread equally on the back of the leaf of the acclimated plant. Since inoculating the Agrobacterium tumefaciens on the whole leaf caused the leaf to get weighted, it was likely for the plant to collapse or for leaf cushion to be broken. Therefore, it was carefully inoculated. The plant introduced with agrobacterium returned to its original state in 10 min-1 hr. The plant introduced with agrobacterium was harvested in 3-5 days and analyzed using the method described in Example 5

### Example 5

### Expression Analysis of Introduced Gene in Gene-Introduced Plant

The expression of gene obtained in the Example 4 was verified as follows: some leaves were collected to verify the expression of GFP gene, and then, the emission of fluorescence light was observed using a portable UV lamp (long-wavelength 366 nm) in dark room.

If a GFP gene has been expressed, leaf cells introduced with the gene emit fluorescence light under the radiation of UV in dark condition. In the present invention, the leaves introduced with the gene emitted fluorescence light. This result confirmed that the gene was introduced and expressed in the leaves (see, FIG. 5).

### Example 6

### Isolation and Purification of GFP from Gene-Introduced Plant

### Example 6-1

### Isolation and Purification of Protein in a Small Scale

One g of Nicotiana benthamiana leaves introduced with GFP gene was cut and grinded in the mortar with liquid nitrogen. Two ml of extracting buffer (250 mM sucrose, 1 M Hepes, 1 mM MgCl₂ and 1 mM DTT) containing a protease inhibitor per 0.5 g of raw plant weight was added and grinded well. The extract was centrifuged for 30 min at 12,000 rpm and 4° C., followed by collecting supernatant. The supernatant was loaded on the column containing ice crystals, and the column was shaken softly for 30-60 min to allow AFP-GFP protein to be attached to the ice crystals. The ice crystals were collected by centrifugation at 500×g for 5 min.

The collected ice crystals were dissolved in the same amount of phosphate buffer, and the released proteins were concentrated using centricon (Amicon Co., U.S.A.).

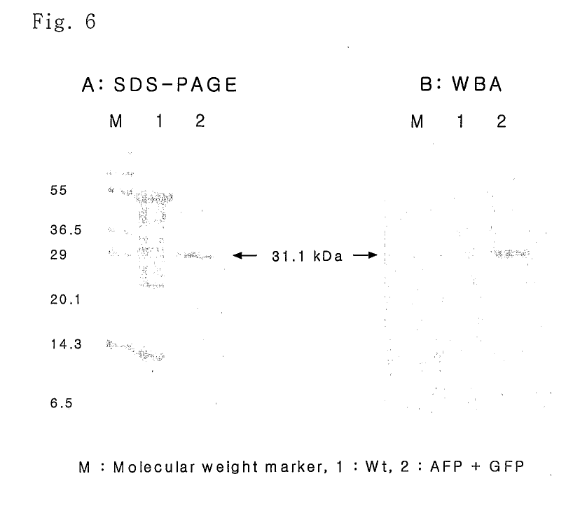

As shown in FIG. 6, AFP-GFP protein, the result of electrophoresis on SDS-PAGE followed by coomassie blue staining confirmed that 31.1 kD of AFP-fused GFP protein was detected in the lysate, but not in wild type of Nicotiana benthamiana.

### Example 6-2

### Isolation and Purification of Protein in a Mass Scale Using Ice-Filled Column

One kg of plant samples prepared by agroinfiltration treatment according Example 4-2 or freeze-preserved after agroinfiltration treatment was pre-cooled into 0-4° C. or thawed. Two ml of extracting buffer (250 mM sucrose, 1 M Hepes, 1 mM MgCl₂ and 1 mM DTT) containing a protease inhibitor per 1.0 g of raw plant weight was added and grinded well. The extract was centrifuged for 30 min at 12,000 rpm and 4° C., followed by collecting supernatant. The supernatant was loaded on the column containing ice crystals, and the column was shaken softly for 30-60 min to allow AFP-GFP protein to be attached to the ice crystals. The ice crystals were collected by centrifugation at 500×g for 5 min.

The collected ice crystals were dissolved in the same amount of phosphate buffer, and the released proteins were concentrated using centricon (Amicon Co., U.S.A.).

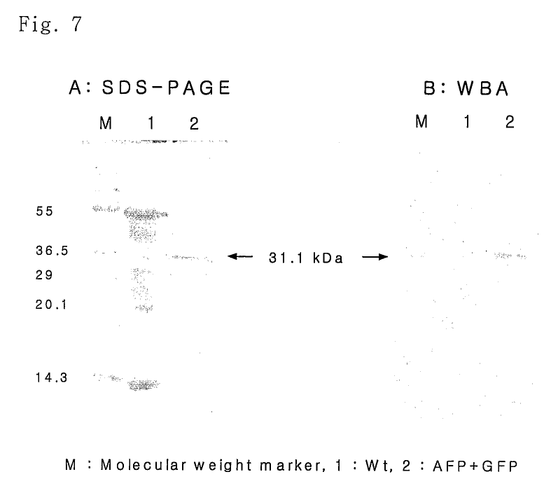

As shown in FIG. 7, AFP-GFP protein, the result of electrophoresis on SDS-PAGE followed by coomassie blue staining confirmed that 31.1 kD of AFP-fused GFP protein was detected in the lysate, but not in wild type of Nicotiana benthamiana.

### Example 6-3

### Isolation and Purification of Protein in a Mass Scale Using Ice-Nucleation Material

One kg of plant samples prepared by agroinfiltration treatment according Example 4-2 or freeze-preserved after agroinfiltration treatment was pre-cooled into 0-4° C. or thawed. Two ml of extracting buffer (250 mM sucrose, 1 M Hepes, 1 mM MgCl₂ and 1 mM DTT) containing a protease inhibitor per 1.0 g of raw plant weight was added and grinded well. The extract was centrifuged for 30 min at 12,000 rpm and 4° C., followed by collecting supernatant. The supernatant was super-cooled into 1.8-7.0° C. below zero under stirring in cold-bath. Insoluble AgI powder was added to the supernatant and it was stirred continuously to allow AFP-GFP protein to be attached to the ice crystals until ⅔ of the supernatant was formed into ice-crystals. The ice formed crystals were collected by centrifugation at 500×g for 5 min.

The collected ice crystals were dissolved in the same amount of phosphate buffer, and the released proteins were concentrated using centricon (Amicon Co., U.S.A.).

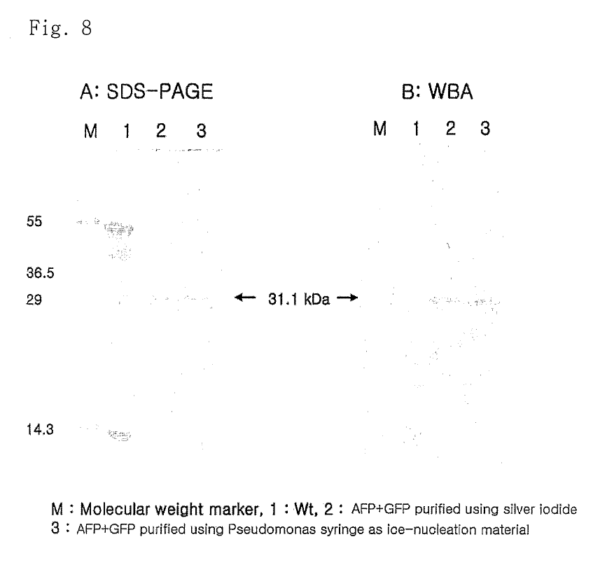

As shown in FIG. 8, AFP-GFP protein, the result of electrophoresis on SDS-PAGE followed by coomassie blue staining confirmed that 31.1 kD of AFP-fused GFP protein was detected in the lysate, but not in wild type of Nicotiana benthamiana.

Alive or dead Pseudomonas syringae may provide the identical result obtained with iodide phosphorus.

### Example 6-4

### Isolation and Purification of Protein in a Mass Scale Using Hypertonic Solution

One kg of plant samples prepared by agroinfiltration treatment according Example 4-2 or freeze-preserved after agroinfiltration treatment was pre-cooled into 0-4° C. or thawed. Two ml of extracting buffer (15% sucrose, 1 M Hepes, 1 mM MgCl₂ and 1 mM DTT) containing a protease inhibitor per 1.0 g of raw plant weight was added and grinded well. The extract was centrifuged for 30 min at 12,000 rpm and 4° C., followed by collecting supernatant. The supernatant was stirred continuously to allow AFP-GFP protein to be attached to the ice crystals in cold-bath until ⅔ of the supernatant was formed into ice-crystals. The ice formed crystals were collected by centrifugation at 500×g for 5 min.

The collected ice crystals were dissolved in the same amount of phosphate buffer, and the released proteins were concentrated using centricon (Amicon Co., U.S.A.).

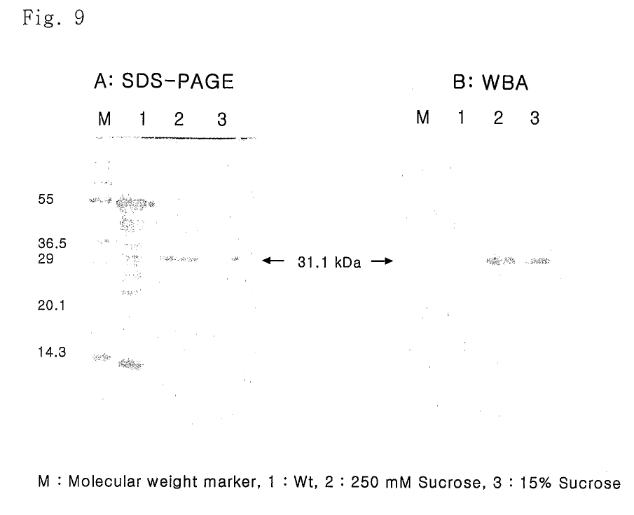

As shown in FIG. 9, the result of electrophoresis on SDS-PAGE followed by coomassie blue staining demonstrated that 31.5 kD of AFP-fused GFP protein was detected in the lysate, but not in wild type of Nicotiana benthamiana.

### Example 6-5

### Isolation and Purification of Protein in a Mass Scale Using Freeze-Control Device

A freeze-control device equipped with a low temperature controller and a stirrer for controlling freezing-rate, for example, a conventional ice slush maker enables us to conveniently isolating and purifying protein in a mass scale according to 6-3 and 6-4.

4500 ml of the extract prepared according to 6-3 and 6-4 was placed in Ice Maker (Sea Corporation) and frozen until ⅔ of the supernatant was formed into ice-crystals for about 70 min. Unfrozen solution was vented and 1000 ml of extracting buffer cooled to 0° C. was added. The ice crystals were washed twice for 30 sec to remove materials except the target protein.

The ice crystals were dissolved in the same amount of phosphate buffer, and the released proteins were concentrated using centricon (Amicon Co., U.S.A.).

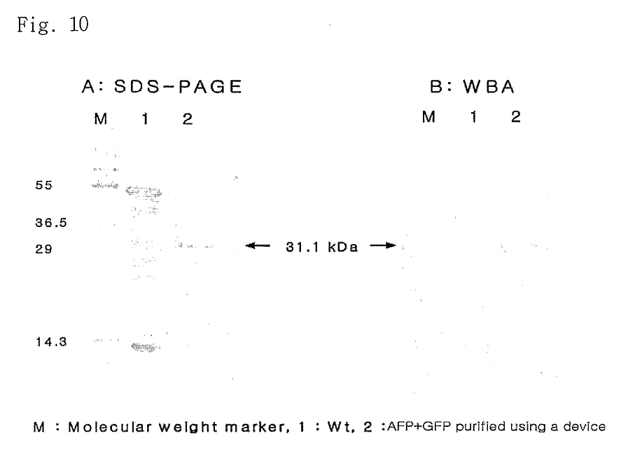

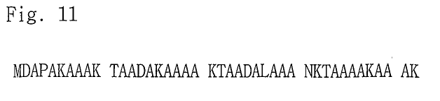

As shown in FIG. 10, the result of electrophoresis on SDS-PAGE followed by coomassie blue staining demonstrated that 31.1 kD of AFP-fused GFP protein was detected in the lysate, but not in wild type of Nicotiana benthamiana.

### Example 7

### Western Blotting Analysis of GFP in Gene-Introduced Plant

The quantification of the recombinant proteins purified by the method of Example 6 was performed in accordance with Bradford method in a manner that dye (protein assay kit, Bio-Rad) was added to protein solution and the absorbance at 595 nm was measured with UV-spectrophotometer, followed by determining the protein amount with reference to the standard curve of bovine serum albumin.

The protein samples in a same concentration were electrophoresed on 8% polyacrylamide gel and were subjected to Western blotting analysis (Peter B. Kaufma et al., Molecular and Cellular Methods in Biology and Medicine, 108-121, CRC press). The Western blotting was performed as follows: Proteins on SDS-PAGE were transferred to PVDF membrane using Semi-Dry Transfer Units (Hoefer, USA) and the PVDF membrane was then dried. The PVDF membrane was incubated for 1 hr in 0.5% BSA/TBS (Tris-buffered saline) containing the primary monoclonal antibody specific to recombinant GFP antigen and washed three times with TBS. Then, the membrane was incubated with the peroxidase-conjugated secondary antibody (peroxidase-labeled rabbit IgG, KPL, USA) for 1 hr and washed with three times with TBS. The color development was allowed with ABTS developer (KPL, USA) for 30 min. As shown in FIGS. 6, 7, 8, 9 and 10, the 31.1 kD band corresponding to recombinant GFP was observed.

Having described specific examples of the present invention, it is to be understood that such examples are only preferred embodiments and should not be construed as limiting the scope of the invention. Therefore, the substantive scope of the invention may be determined by appended claims and their equivalents

### INDUSTRIAL APPLICATION

As described above, the present invention relates to a method for the production, isolation, and purification of a recombinant protein, more particularly, to a method for isolating and purifying a foreign protein stably using Anti-Freeze Protein (AFP), thereby producing the protein. The present invention provides a method for the production, isolation and purification of a foreign target protein using its recombinant protein containing AFP, and a construct, an expression vector, a transformant and a recombinant protein. The recombinant protein produced by the present invention shows the biological property and function identical to a naturally occurring protein. Particularly, the present invention is advantageous for the expression and purification of a useful protein.

1. A polynucleotide encoding anti-freeze protein, comprising a nucleotide sequence represented by SEQ ID NO:1.

2. A nucleotide construct composed, in the following order, of a nucleotide sequence encoding anti-freeze protein comprising nucleotide sequence represented by SEQ ID NO:1, protease cleavage site, multiple cloning site comprising sites recognized by plural restriction enzymes, and stop codon.

3. A nucleotide construct composed, in the following order, of multiple cloning site comprising sites recognized by plural restriction enzymes, protease cleavage site, a nucleotide sequence encoding anti-freeze protein comprising nucleotide sequence represented by SEQ ID NO:1, and stop codon.

4. The nucleotide construct according to claim 2, wherein said multiple cloning site comprises at least two recognition sites selected from the group consisting of NcoI, XbaI, and BamHI.

5. The nucleotide construct according to claim 2, wherein said protease cleavage site is enterokinase cleavage site.

6. The nucleotide construct according to claim 3, wherein said protease cleavage site is thrombin cleavage site.

7. The nucleotide construct according to claim 2, wherein said stop codon is TAG.

8. The nucleotide construct according to claim 2, wherein said nucleotide construct comprises a nucleotide sequence represented by SEQ ID NO:2.

9. The nucleotide construct according to claim 3, wherein said nucleotide construct comprises a nucleotide sequence represented by SEQ ID NO:3.

10. An expression vector for plant comprising (i) the nucleotide construct according to claim 2, wherein a nucleotide sequence encoding a target protein is inserted into the multiple cloning site; (ii) a promoter that functions in plant cells to cause the production of an RNA molecule operably linked to the nucleotide construct of (i); and (iii) a 3′-non-translated region that functions in plant cells to cause the polyadenylation of the 3′-end of said RNA molecule.

11. A method for preparing a transient transfected plant expressing a recombinant protein transiently, which comprises the steps of:
(a) introducing the plant expression vector according to claim 10 into a plant cell; and
(b) confirming whether the gene has been introduced into said plant cell.

12. A transient transfected plant prepared by the method according to claim 11, expressing the recombinant protein transiently.

13. A method for producing a recombinant protein by using a transient transgenic plant as a bioreactor, which comprises the steps of:
(a) introducing the plant expression vector according to claim 10 into a plant cell;
(b) confirming whether the gene has been introduced into said plant cell; and
(c) obtaining the recombinant protein from a plant comprising the plant cell introduce with the gene.

14. A method for preparing a transgenic plant expressing a recombinant protein stably, which comprises the steps of:
(a) introducing the expression vector for plant according to claim 10 into a plant cell;
(b) selecting a transformed plant cell; and
(c) regenerating a plant from the transformed plant cell to obtain a transgenic plant.

15. A transgenic plant prepared by the method according to claim 14, expressing the recombinant protein stably.

16. A method for producing a recombinant protein by using a transgenic plant as a bioreactor, which comprises the steps of:
(a) introducing the expression vector for plant according to claim 10 into a plant cell;
(b) selecting a transformed plant cell;
(c) regenerating a plant from the transformed plant cell to obtain a transgenic plant; and
(d) obtaining the recombinant protein from the transgenic plant.

17. A recombinant protein produced by the method according to claim 13.

18. The method according to claim 13, said step of obtaining the recombinant protein is performed by using an ice-filled column.

19. The method according to claim 13, said step of obtaining the recombinant protein is performed by using an ice-nucleation material comprising silver iodide crystal or alive or dead microorganism, Pseudomonas syringae.

20. The method according to claim 13, said step of obtaining the recombinant protein is performed by using a hypertonic solution comprising monosaccharides, disaccharides, polysaccharides or sugar-alcohol.

21. The method according to claim 13, said step of obtaining the recombinant protein is performed by using a freeze-control device equipped with a low temperature controller and a stirrer, capable of controlling freezing-rate.

22. The method according to claim 19, wherein said method further uses a freeze-control device equipped with equipped a low temperature controller and a stirrer, capable of controlling freezing-rate.

23. A method for isolating AFP-fused recombinant protein, which comprises the step of;
(a) contacting to ice crystal a recombinant fusion protein comprising target protein and AFP; and
(b) recovering the ice crystal to which the recombinant protein is attached.

24. The method according to claim 23, wherein said AFP is derived from plants, fungi or fishes.

25. The method according to claim 23, said AFP corresponds to the ice crystal-attaching domain of the full length of AFP amino acid sequence.

26. The method according to claim 23, wherein said recombinant protein is produced by the method for preparing a transgenic plant expressing the recombinant protein, which comprises the steps of;
(a) preparing an expression vector comprising a construct in which a nucleotide sequence encoding AFP are linked to 5′-end or 3′-end of a nucleotide sequence encoding a target protein and protease cleavage site exists between the target protein-coding sequence and AFP-coding sequence;
(b) introducing the expression vector into a host; and
(c) selecting a transformed host.

27. The method according to claim 26, wherein said protease cleavage site is enterokinase cleavage site.

28. The method according to claim 26, wherein said expression vector is an expression vector for plant, animal or microorganism.

29. The method according to claim 26, wherein said host is a cell of plant, animal or microorganism, a plant or an animal.


SEQUENCE LISTING (3 sequences)

SEQ ID NO: 1 - DNA, 126 bp, Artificial Sequence
  optimized AFP nucleotide sequence
atggatgctccagctaaagcagcagcaaaaacagctgcagatgcaaaagctgctgctgctaaaactgcagcagatgcattagctgctgctaataaaactgcagcagctgctaaagcagctgcaaaa

SEQ ID NO: 2 - DNA, 165 bp, Artificial Sequence
  AFPN nucleotide sequence comprising AFP nucleotide sequence
ccatggatgctccagctaaagcagcagcaaaaacagctgcagatgcaaaagctgctgctgctaaaactgcagcagatgcattagctgctgctaataaaactgcagcagctgctaaagcagctgcaaaagacgacgacgacaaggctctagaggatccatagatct

SEQ ID NO: 3 - DNA, 171 bp, Artificial Sequence
  AFPC nucleotide sequence comprising AFP nucleotide sequence
ccatggctctagaggatcccctcgttccacgaggatctatggatgctccagctaaagcagcagcaaaaacagctgcagatgcaaaagctgctgctgctaaaactgcagcagatgcattagctgctgctaataaaactgcagcagctgctaaagcagctgcaaaatagatct